(19) United States
(12) Patent Application Publication
(10) Pub. No.: US 2020/0397684 A1
(43) Pub. Date: Dec. 24, 2020

(54) PROCESS FOR DYEING HAIR

(71) Applicant: Henkel AG & Co. KGaA, Duesseldorf (DE)

(72) Inventors: TORSTEN LECHNER, Langenfeld (DE); JUERGEN SCHOEPGENS, Schwalmtal (DE); MARC NOWOTTNY, Moenchengladbach (DE)

(73) Assignee: Henkel AG & Co. KGaA, Duesseldorf (DE)

(21) Appl. No.: 16/977,084

(22) PCT Filed: Feb. 7, 2019
(86) PCT No.: PCT/EP2019/052975

§ 371 Date: Aug. 31, 2020

(30) Foreign Application Priority Data
Mar. 14, 2018 (DE) 10 2018 203 838.4

(51) Int. Cl. A61K 8/58; A61K 8/25; A61K 8/29; A61Q 5/10

(52) U.S. Cl. CPC A61K 8/585; A61K 8/25; A61K 8/29; A61K 2800/884; A61K 2800/30; A61K 2800/4324; A61Q 5/10

(57) ABSTRACT

The subject of the present disclosure is a process for dyeing human hair, in which
a pretreatment agent (A) which contains at least one organic silicon compound of the formula (I) and/or (II) and contains no direct dyes and no pigments, and
a colorant (B) which contains at least one organic silicon compound of the formula (I) and/or (II) and further contains at least one colorant compound from the group of direct dyes and/or pigments, can be applied to the hair,
   wherein the organic silicon compounds of formulae (I) and (II) are defined as follows

R₁R₂N-L-Si(OR₃)ₐ(R₄)_{b} (I),

(R₅O)_{c}(R₆)_{d}Si-(A)ₑ-[NR₇-(A′)]_{f}—[O-(A″)]_{g}-[NR₈-(A′″)]ₕ—Si(R₆′)_{d′}(OR₅′)_{c′} (II).

### CROSS-REFERENCE TO RELATED APPLICATION

This application is a U.S. National-Stage entry under 35 U.S.C. § 371 based on International Application No. PCT/EP2019/052975, filed Feb. 7, 2019, which was published under PCT Article 21(2) and which claims priority to German Application No. 10 2018 203 838.4, filed Mar. 14, 2018, which are all hereby incorporated in their entirety by reference.

### TECHNICAL FIELD

The subject-matter of the present application is a process for coloring human hair, in which two different agents, a pretreatment agent (A) and a coloring agent (B), are applied to the hair. The pretreatment agent (A) is free from coloring compounds and contains at least one special organic silicon compound of formula (I) and/or (II). The colorant (B) also contains at least one organic silicon compound of the formula (I) and/or (II) and additionally at least one colorant compound from the group of direct dyes and/or pigments.

Another subject matter of this notification is a kit-of-parts for coloring human hair, which contains separately packed in different containers an agent (A), an agent (B) and possibly another agent (B′). These agents are used to produce the pre-treatment agent (A) and the colorant (B) described above.

### BACKGROUND

The change in shape and color of keratin fibers, especially hair, is an important area of modern cosmetics. To change the hair color, the expert knows various coloring systems depending on coloring requirements. Oxidation dyes are usually used for permanent, intensive dyeing's with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the dye into the hair fiber. Compared to oxidative hair dyeing, the dyeing's obtained with direct dyes have a shorter shelf life and quicker wash ability. Dyeing's with direct dyes usually remain on the hair for a period of between about 5 and about 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. They can therefore be removed again without residue by a few washes with detergents containing surfactants. Various products of this type are available on the market under the name hair mascara.

If the user wants particularly long-lasting dyeing's, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

### BRIEF SUMMARY

Process for dyeing human hair and multi-component packaging units are provided herein. In an embodiment, a process for dyeing human hair is provided, wherein
a pretreatment agent (A) which comprises at least one organic silicon compound of the formula (I) and/or (II) and comprises no direct dyes and no pigments, and
a colorant (B) which comprises at least one organic silicon compound of the formula (I) and/or (II) and further comprises at least one colorant compound from the group of direct dyes and/or pigments,
   is applied to the hair.
   In the organic silicon compound of formula (I)

R₁R₂N-L-Si(OR₃)ₐ(R₄)_{b} (I),
R₁, R₂ independently of one another represent a hydrogen atom or a C₁-C₆ alkyl group,
L is a linear or branched divalent C₁-C₂₀ alkylene group,
R₃, R₄ independently of one another represent a C₁-C alkyl group,
a, is an integer from 1 to 3, and
b stands for the integer 3−a.
   In the organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
R5, R5′, R5″, R6, R6′ and R6″ independently of one another represent a C₁-C₆ alkyl group,
A, A′, A″, and A′″ independently of one another represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A′″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
represents a linear or branched divalent C₁-C₂₀ alkylene group,
provided that at least one of e, f, g, and h is different from 0.

In anther embodiment, a multi-component packaging unit for dyeing human hair includes, comprehensively assembled separately
a first container with an agent (A) which comprises at least one organic silicon compound of the formula (I) and/or (II) and comprises no direct dyes and no pigments, and a second container with an agent (B) comprising at least one organic silicon compound of formula (I) and/or (II), and
optionally, a third container with a medium (B′).
   At least one of the agents (B) and/or (B′) comprises at least one colorant compound from the group of direct dyes and pigments. In the organic silicon compound of formula (I)

R₁R₂N-L-Si(OR₃)ₐ(R₄)_{b} (I),
R₁, R₂ independently of one another represent a hydrogen atom or a C₁-C₆ alkyl group,
L is a linear or branched divalent C₁-C₂₀ alkylene group,
R₃, R₄ independently of one another represent a C₁-C alkyl group,
a, is an integer from 1 to 3, and
b stands for the integer 3−a.
   In the organic silicon compound of formula (II)

(R₅O)_{c}(R₆)_{d}Si-(A)_{c}-[NR₇-(A′)]_{f}—[O-(A″)]_{g}—[NR₈-(A′″)]ₕ—Si(R₆′)_{d′}(OR₅′)_{c′} (II),
R5, R5′, R5″, R6, R6′ and R6″ independently of one another represent a C₁-C₆ alkyl group,
A, A′, A″, and A′″ independently of one another represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A′″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
A′ represents a linear or branched divalent C₁-C₂₀ alkylene group,
provided that at least one of e, f, g, and h is different from 0.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The purpose of the present disclosure was to provide a dyeing system with fastness properties comparable to those of oxidative dyeing. Wash fastness properties should be outstanding, but the use of oxidation dye precursors normally used for this purpose should be avoided. A technology was sought that would allow the direct dyes and pigments known from state-of-the-art technology to be fixed to the hair in an extremely permanent manner

Surprisingly, it has now turned out that the above-mentioned task can be excellently solved if the hair is dyed using a process in which first a first pretreatment agent (A) is applied to the hair and then the actual dye (B) is applied to the hair. The pretreatment agent (A) does not itself contain any dyes or coloring compounds. A characteristic feature of the pretreatment agent (A) is its content of at least one reactive organic silicon compound of the formula (I) and/or (II). The reactive organic silicon compound(s) functionalize the hair surface as soon as they meet it. In this way a first, still uncolored film is formed. In the second step of the process, a colorant (B) is now applied to the hair. This also contains at least one reactive organic silicon compound of the formula (I) and/or (II), and additionally at least one coloring compound from the group of direct dyes and/or pigments. During the application of the dye (B) to the hair, a film is also formed on the—now already functionalized—hair surface, whereby the coloring compounds are now embedded in the film and thus deposited on the hair. The film produced “in situ” in this way, in which the coloring compound is embedded, is exemplified by outstanding wash fastness.

A first subject matter of the present disclosure is a process for dyeing human hair, in which
a pretreatment agent (A) which contains at least one organic silicon compound of the formula (I) and/or (II) and contains no direct dyes and no pigments, and
a colorant (B) which contains at least one organic silicon compound of the formula (I) and/or (II) and further contains at least one colorant compound from the group of direct dyes and/or pigments,
   can be applied to the hair,
   wherein in the organic silicon compound of formula (I)

R₁R₂N-L-Si(OR₃)ₐ(R₄)_{b} (I),
R₁, R₂ independently of one another represent a hydrogen atom or a C₁-C₆ alkyl group,
L is a linear or branched divalent C₁-C₂₀ alkylene group,
R₃, R₄ independently of one another represent a C₁-C₆ alkyl group,
a, is an integer from 1 to 3, and
b stands for the integer 3−a, and
   wherein in the organic silicon compound of formula (II)

(R₅O)_{c}(R₆)_{d}Si-(A)_{c}-[NR₇-(A′)]_{f}—[O-(A′″)]_{g}—[NR₈-(A′″)]ₕ—Si(R₆′)_{d′}(OR₅′)_{c′} (II),
R5, R5′, R5″, R6, R6′ and R6″ independently of one another represent a C₁-C₆ alkyl group,
A, A′, A″, A′″ and A′″ independently of one another represent a linear or branched divalent C₁-C₂₀ alkylene group
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g, and h is different from 0.

### Process for Dyeing Human Hair

As contemplated herein, human hair is understood to be the human head hair. The term “coloring agent” is used in the context of this present disclosure for a coloring of the keratin fibers, in particular the hair, caused by the use of direct dyes and/or color pigments, the aforementioned coloring compounds being deposited in a film on the surface of the keratin material. The film is formed in situ by oligomerization or polymerization of the organic silica compound or compounds of formula (I) and/or (II) initiated by their contact with water.

Due to the high resistance of the formed film, the produced dyeing's have an extremely good water resistance and wash fastness.

In the process as contemplated herein, the pretreatment agent (A) and the colorant (B) are applied to the hair.

In a first step, the pretreatment agent (A) is first applied to the hair, whereby the pretreatment agent (A) is the pretreatment agent (A) ready for use. The dye (B) is then applied to the hair. The colorant (B) is also the ready-to-use colorant (B).

The pretreatment agent (A) (which is the ready-to-use pretreatment agent) can, for example, be formulated based on an aqueous, or aqueous-alcoholic carrier. Suitable forms of packaging are for example a cream, an emulsion, a gel, or a surfactant-containing foaming solution such as a shampoo or foam aerosol.

The colorant (B) (which is the ready-to-use colorant) can also be formulated based on an aqueous or aqueous-alcoholic carrier, for example. Suitable forms of packaging are for example a cream, an emulsion, a gel, or a surfactant-containing foaming solution such as a shampoo or foam aerosol.

In addition to the application of the pretreatment agent (A) and the colorant (B), the method as contemplated herein may also include the application of a further agent, which may for example be an after-treatment agent (C). By applying the after-treatment agent (C), the hair can be cleaned and/or cared for, for example, in a final process step.

Within the scope of one version, a method for dyeing human hair is thus preferred, in which
a pretreatment agent (A) which contains at least one organic silicon compound of the formula (I) and/or (II) and contains no direct dyes and no pigments, and
a colorant (B) which contains at least one organic silicon compound of the formula (I) and/or (II) and further contains at least one colorant compound from the group of direct dyes and/or pigments, and
an after-treatment agent (C) containing at least one cleansing and/or conditioning active substance,
   can be applied to the hair,
   wherein in the organic silicon compound of formula (I)

R₁R₂N-L-Si(OR₃)ₐ(R₄)_{b} (I),
R₁, R₂ independently of one another represent a hydrogen atom or a C₁-C₆ alkyl group,
L is a linear or branched divalent C₁-C₂₀ alkylene group,
R₃, R₄ independently of one another represent a C₁-C₆ alkyl group,
a, is an integer from 1 to 3, and
b stands for the integer 3−a, and
   wherein in the organic silicon compound of formula (II)

(R₅O)_{c}(R₆)_{d}Si-(A)ₑ-[NR₇-(A′)]_{f}-[O-(A′″)]_{g}—[NR₈-(A′″)]ₕ—Si(R₆′)_{d′}(OR₅′)_{c′} (II),
R5, R5′, R5″, R6, R6′ and R6″ independently of one another represent a C₁-C₆ alkyl group,
A, A′, A″, A′″ and A′″ independently of one another represent a linear or branched divalent C₁-C₂₀ alkylene group
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g, and h is different from 0.

### Pretreatment Agent (A)

In the first step of the process as contemplated herein, the pretreatment agent (A) is applied to the hair. The pretreatment agent (A) itself does not color the hair, i.e. it is free of coloring compounds from the group of direct dyes and pigments.

In the context of a preferred embodiment, the method as contemplated herein is exemplified in that
a pretreatment agent (A) which contains at least one organic silicon compound of the formula (I) and/or (II) and is further free from coloring compounds,
   is applied to the hair.

In the context of a preferred embodiment, the method as contemplated herein is exemplified in that
a pretreatment agent (A) which contains at least one organic silicon compound of the formula (I) and/or (II) and further contains no direct dyes and no pigments and no oxidation dye precursors,
   is applied to the hair.

Direct dyes are those dyes that are usually used by professionals in the direct dyeing of hair. Common direct dyes are disclosed in the description of the colorant (B). Pigments are those coloring pigments that are usually used by specialists for temporary hair coloring. Common pigments or color pigments are disclosed in the description of the colorant (B). All these coloring compounds are not contained in pretreatment agent (A), i.e. the total amount of coloring compounds from the group of direct dyes and pigments contained in pretreatment agent (A) is—based on the total weight of pretreatment agent (A)—below 0.05 wt. %, preferably below 0.01 wt. % and particularly preferably below 0.001 wt. %.

The pretreatment agent (A) contains at least one organic silicon compound of formula (I) and/or (II),

R₁R₂N-L-Si(OR₃)ₐ(R₄)_{b} (I),

wherein in the organic silicon compound of formula (I)
R₁, R₂ independently of one another represent a hydrogen atom or a C₁-C₆ alkyl group,
L is a linear or branched divalent C₁-C₂₀ alkylene group,
R₃, R₄ independently of one another represent a C₁-C₆ alkyl group,
a, is an integer from 1 to 3, and
b stands for the integer 3−a, and

(R₅O)_{c}(R₆)_{d}Si-(A)_{c}-[NR₇-(A′)]_{f}—[O-(A′)]_{g}—[NR₈-(A′″)]ₕ—Si(R₆′)_{d′}(OR₅′)_{c′} (II),

wherein in the organic silicon compound of formula (II)
R5, R5′, R5″, R6, R6′ and R6″ independently of one another represent a C₁-C₆ alkyl group,
A, A′, A″, A′″ und A′″ independently of one another represent a linear or branched divalent C₁-C₂₀ alkylene group
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g, and h is different from 0.

Organic silicon compounds, alternatively called organosilicon compounds, are compounds which either have a direct silicon-carbon bond (Si—C) or in which the carbon is bonded to the silicon atom via an oxygen, nitrogen, or sulfur atom.

The substituents R₁, R₂, R₃, R₄, R₅, R₅′, R₅″, R₆, R₆′, R₆″, R₇, R₈, L, A, A′, A″, A′″ und A′″ in the compounds of formula (I) and (II) are explained below by way of example: Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆-alkenyl group are vinyl, allyl, but-2-enyl, but-3-enyl and isobutenyl, preferred C₂-C₆-alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl, a 5-hydroxypentyl and a 6-hydroxyhexyl group; a 2-hydroxyethyl group is particularly preferred. Examples of an amino-C₁-C₆-alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂—), the ethylene group (—CH₂—CH₂—), the propylene group (—CH₂—CH₂—CH₂) and the butylene group (—CH₂—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R1 and R2 independently of one another represent a hydrogen atom or a C₁-C₆ alkyl group. In particular, the radicals R1 and R2 both represent a hydrogen atom.

In the middle part of the organic silicon compound is the structural unit or the linker -L- which stands for a linear or branched, divalent C₁-C₂₀ alkylene group.

Preferably -L- stands for a linear, divalent C₁-C₂₀ alkylene group. Further preferably -L- stands for a linear divalent C₁-C₆ alkylene group. Particularly preferred -L- stands for a methylene group (—CH₂—), an ethylene group (—CH₂—CH₂—), a propylene group (—CH₂—CH₂—CH₂—) or a butylene group (—CH₂—CH₂—CH₂—CH₂—). L stands for a propylene group (—CH₂—CH₂—CH₂—).

A process which is particularly preferred within the framework of a further version is exemplified in that the pretreatment agent (A) contains at least one organic silicon compound of formula (I),

R₁R₂N-L-Si(OR₃)ₐ(R₄)_{b} (I),

where

R₁, R₂ both represent a hydrogen atom, and

L represents a linear, divalent C₁-C₆ alkylene group, preferably a propylene group (—CH₂—CH₂—CH₂—) or an ethylene group (—CH₂—CH₂—).

In the context of a further embodiment, a process is particularly preferred which is exemplified in that in the organic silicon compound of the formula (I)

R₁R₂N-L-Si(OR₃)ₐ(R₄)_{b} (I),

R₁, R₂ both represent a hydrogen atom, and

L represents a linear, divalent C₁-C₆ alkylene group, preferably a propylene group (—CH₂—CH₂—CH₂—) or an ethylene group (—CH₂—CH₂—).

The organic silicon compounds of formula (I)

R₁R₂N-L-Si(OR₃)ₐ(R₄)_{b} (I),

one end of each carries the silicon-containing group —Si(OR₃)ₐ(R₄)_{b},

In the terminal structural unit —Si(OR₃)ₐ(R₄)_{b}, the radicals R3 and R4 independently of one another represent a C₁-C₆ alkyl group. Particularly preferably, R3 and R4 independently of one another represent a methyl group or an ethyl group.

Here a stand for an integer from 1 to 3, and b stands for the integer 3−a. If a stand for the number 3, then b is equal to 0. If a stand for the number 2, then b is equal to 1. If a stand for the number 1, then b is equal to 2.

Dyes with the best wash fastness values could be obtained if the pretreatment agent contains at least one organic silicon compound corresponding to formula (I): in which R3, R4 independently of one another represent a methyl group or an ethyl group.

Dyes with the best wash fastness values could be obtained if the pretreatment agent contains at least one organic silicon compound of the formula (I) in which the radical a represents the number 3. In this case the rest b stands for the number 0.

A process which is particularly preferred within the framework of a further version is exemplified in that the pretreatment agent (A) contains at least one organic silicon compound of formula (I),

R₁R₂N-L-Si(OR₃)ₐ(R4)_{b} (I),

where
R3, R4 independently of one another represent a methyl group or an ethyl group and
a stands for the number 3 and
b stands for the number 0.

In the context of a further embodiment, a process is particularly preferred which is exemplified in that in the organic silicon compound of the formula (I)

R₁R₂N-L-Si(OR₃)ₐ(R₄)_{b} (I),
R3, R4 independently of one another represent a methyl group or an ethyl group and
a stands for the number 3 and
b stands for the number 0.

Organic silicon compounds of the formula (I) which are particularly suitable for solving the problem as contemplated herein are

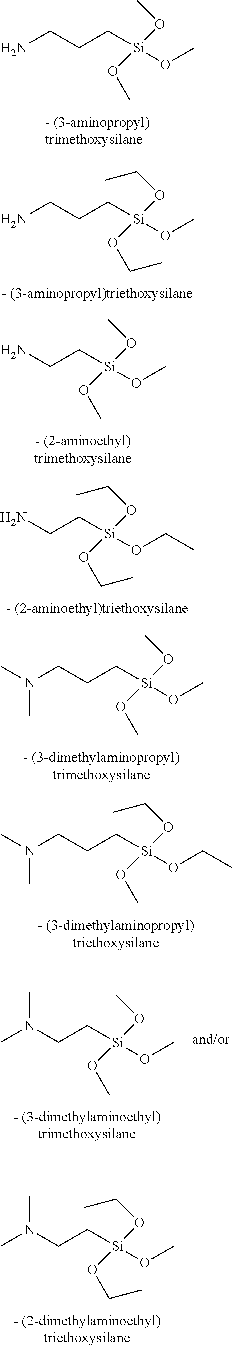

In a further preferred embodiment, a process as contemplated herein is exemplified in that the pretreatment agent (A) contains at least one organic silicon compound of formula (I) which is selected from the group of
(3-aminopropyl)trimethoxysilane
(3-aminopropyl)triethoxysilane
(2-aminoethyl)trimethoxysilane
(2-aminoethyl)triethoxysilane
(3-dimethylaminopropyl)trimethoxysilane
(3-dimethylaminopropyl)triethoxysilane
(2-dimethylaminoethyl)trimethoxysilane and/or
(2-dimethylaminoethyl)triethoxysilane.

In the context of a further version, a process is particularly preferred in which the organic silicon compound of the formula (I) is selected from the group of
(3-aminopropyl)trimethoxysilane
(3-aminopropyl)triethoxysilane
(2-aminoethyl)trimethoxysilane
(2-aminoethyl)triethoxysilane
(3-dimethylaminopropyl)trimethoxysilane
(3-dimethylaminopropyl)triethoxysilane
(2-dimethylaminoethyl)trimethoxysilane and/or
(2-dimethylaminoethyl)triethoxysilane.

The organic silicon compound of formula (I) is commercially available. (3-aminopropyl)trimethoxysilane, for example, can be purchased from Sigma-Aldrich. Also (3-aminopropyl)triethoxysilane is commercially available from Sigma-Aldrich.

The pretreatment agent (A) may also contain at least one organic silicon compound of formula (II).

The organosilicon compounds of formula (II) as contemplated herein each carry the silicon-containing groups (R₅O)_{c}(R₆)_{d}Si— and —Si(R₆′)_{d′}(OR₅′)_{c′}. at both ends.

In the central part of the molecule of formula (II) there are the groups -(A)ₑ- and —[NR₇-(A′)]_{f}-

and [O-(A″)]_{g}- and -[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) as contemplated herein contains at least one grouping from the group of -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c} the radicals R5, R6, R5′ and R6′ independently of one another represent a C₁-C₆ alkyl group. R5, R6, R5′ and R6′ independently of one another represent a methyl group or an ethyl group.

Here c stands for an integer from 1 to 3, and d stands for the integer 3−c. If c is the number 3, then d is equal to 0. If c stands for the number 2, then d is equal to 1. If c stands for the number 1, then d is equal to 2.

Analogously, c′ stands for an integer from 1 to 3, and d′ stands for the integer 3−a′. If c′ stands for the number 3, then d′ is equal to 0. If c′ stands for the number 2, then d′ is equal to 1. If c′ stands for the number 1, then d′ is equal to 2.

Dyeing's with the best wash fastness values could be obtained if the residues c and c′ both stand for the number 3. In this case d and d′ both stand for the number 0.

In a further preferred embodiment, a process as contemplated herein is exemplified in that the pretreatment agent (A) contains at least one organic silicon compound of formula (II),

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
R5 and R5′ independently represent a methyl group or an ethyl group,
c and c′ both stand for the number 3 and
d and d′ both stand for the number 0

In the context of a further version, a process is particularly preferred which is exemplified in that in the organic silicon compound of the formula (II)

(R₅O)ₑ(R₆)_{d}Si-(A)ₑ-[NR₇-(A′)]_{f}—[O-(A′″)]_{g}—[NR₈-(A′″)]ₕ—Si(R₆′)_{d′}(OR₅′)_{c′} (II),
R5 and R5′ independently represent a methyl group or an ethyl group,
c and c′ both stand for the number 3 and
d and d′ both stand for the number 0

If c and c′ are both the number 3 and d and d′ are both the number 0, the organic silicon compound of the present disclosure corresponds to formula (IIa)

(R₅O)₃Si-(A)ₑ-[NR₇-(A′)]_{f}—[O-(A″)]_{g}—[NR₈-(A′″)]ₕ—Si(OR₅′)₃ (IIa).

The radicals e, f, g, and h can independently stand for the number 0 or 1, whereby at least one of e, f, g, and h is different from zero. The abbreviations e, f, g and h thus define which of the groupings -(A)ₑ- and —[NR₇-(A′)]_{f}- and —[O-(A″)]_{g}- and —[NR₈-(A′″)]ₕ- are located in the middle part of the organic silicon compound of formula (II).

In this context, the presence of certain groupings has proved to be particularly beneficial in terms of increasing washability. Particularly good results were obtained when at least two of the residues e, f, g, and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]—Si(R₆′)_{d′}(OR₅′)_{c′} (IIb).

The radicals A, A′, A″, A′″ and A″″ independently of one another represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently of one another represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂) or a butylene group (—CH₂—CH₂—CH₂—CH₂—). In particular, the radicals A, A′, A″, A′″ and A″″ stand for a propylene group (—CH₂—CH₂—CH₂—).

If the radical f represents the number 1, then the organic silicon compound of formula (II) as contemplated herein contains a structural grouping —[NR₇-(A′)]-. If the radical f represents the number 1, then the organic silicon compound of formula (II) as contemplated herein contains a structural grouping —[NR₈-(A′″)]-.

Here, the radicals R7 and R₈ independently of one another represent a hydrogen atom, a C₁-C₆-alkyl group, a hydroxy-C₁-C₆-alkyl group, a C₂-C₆-alkenyl group, an amino-C₁-C₆-alkyl group or a grouping of the Formula (III)

-(A″″)-Si(R₆″)_{d}″(OR₅″)_{c}″ (III).

Very preferably the radicals R7 and R8 independently of one another represent a hydrogen atom, a methyl group, a 2-hydroxyethyl group, a 2-alkenyl group, a 2-aminoethyl group or a grouping of the formula (III).

If the radical f represents the number 1 and the radical h represents the number 0, the organic silicon compound as contemplated herein contains the grouping [NR₇-(A′)] but not the grouping —[NR₈-(A′″)]. If the radical R7 now stands for a grouping of the formula (III), the pretreatment agents (A) contains an organic silicon compound with 3 reactive silane groups.

In a further preferred embodiment, a process as contemplated herein is exemplified in that the pretreatment agent (A) contains at least one organic silicon compound of formula (II), where
e and f both stand for the number 1,
g and h both stand for the number 0,
A and A′ independently of one another represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In the context of a further version, a process is particularly preferred which is exemplified in that in the organic silicon compound of the formula (II)
e and f both stand for the number 1,
g and h both stand for the number 0,
A and A′ independently of one another represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In a further preferred embodiment, a process as contemplated herein is exemplified in that the pretreatment agent (A) contains at least one organic silicon compound of formula (II), where
e and f both stand for the number 1,
g and h both stand for the number 0,
A and A′ independently of one another represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In a further preferred embodiment, a process as contemplated herein is exemplified in that the pretreatment agent (A) contains at least one organic silicon compound of formula (II), where
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R7 represents a hydrogen atom, a methyl group, a 2-hydroxyethyl group, a 2-alkenyl group, a 2-aminoethyl group or a group of formula (III).

Organic silicon compounds of the formula (II) which are particularly suitable for solving the problem as contemplated herein are

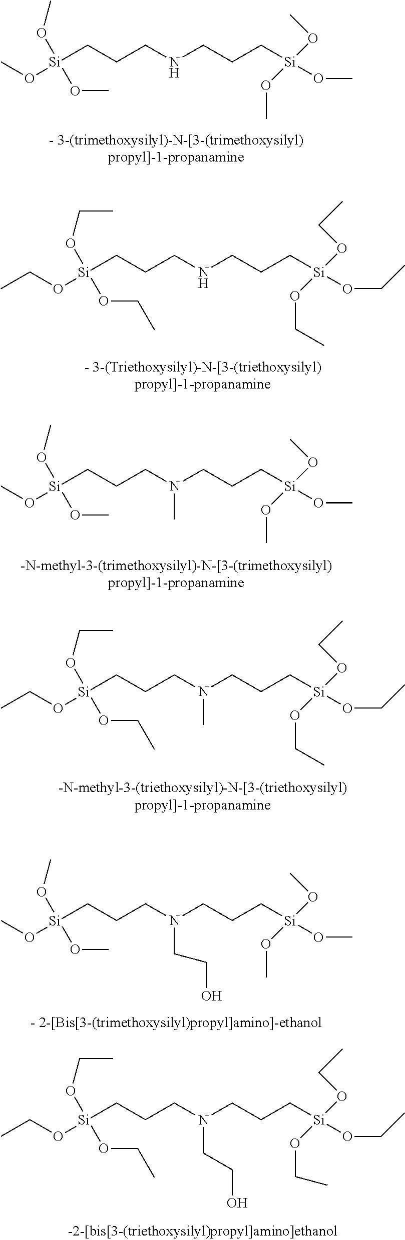

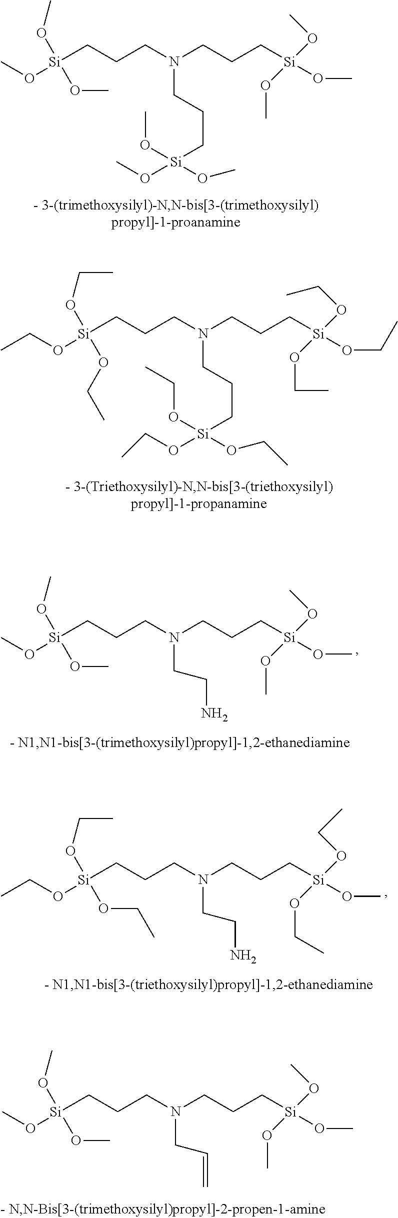

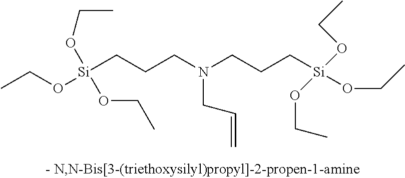 [SMILES: C=CCN(CCC[Si](OCC)(OCC)OCC)CCC[Si](OCC)(OCC)OCC]

The organic silicon compound of formula (II) is commercially available.

For example, Bis(trimethoxysilylpropyl)amines with the CAS number 82985-35-1 can be purchased from Sigma-Aldrich. Bis[3-(triethoxysilyl)propyl]amine with the CAS number 13497-18-2 can be purchased from Sigma-Aldrich, for example. N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorophen. 3-(triethoxysilyl)-N,N-bis[3-(triethoxysilyl)propyl]-1-propanamine with the CAS number 18784-74-2 can be purchased for example from Fluorochem or Sigma-Aldrich.

In a further preferred embodiment, a process as contemplated herein is exemplified in that the pretreatment agent (A) contains at least one organic silicon compound of formula (II) which is selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

In the context of a further version, a process is particularly preferred which is exemplified in that the organic silicon compound of formula (II) is selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

The organic silicon compounds of formula (I) and (II) are reactive compounds. In this context, it has proved to be particularly preferred if the pretreatment agent (A)—based on the total weight of the pretreatment agent (A)—contains one or more organic silicon compounds of the formula (I) and/or (II) in a total amount of from about 0.1 to about 20.0% by weight, preferably from about 0.2 to about 15.0% by weight and particularly preferably from about 0.2 to about 2.0% by weight.

In a further preferred embodiment, a process as contemplated herein is exemplified in that the pretreatment agent (A)—based on the total weight of the pretreatment agent (A)—contains one or more organic silicon compounds of the formula (I) and/or (II) in a total amount of from about 0.1 to about 20.0% by weight, preferably from about 0.2 to about 15.0% by weight and particularly preferably from about 0.2 to about 2.0% by weight.

It has proved to be particularly suitable to use at least one organic silicon compound of formula (I) in the pretreatment agent (A).

In a further preferred embodiment, a process as contemplated herein is exemplified in that the pretreatment agent (A) contains at least one organic silicon compound of formula (I).

### Coloring Agent (B)

Also, the colorant (B), which is applied to the hair as part of the process as contemplated herein, contains at least one organic silicon compound of formula (I) and/or (II).

The organic silicon compound of formula (I) and/or (II) are the same group of compounds as disclosed in the description of the pretreatment agent (A).

Here, the pretreatment agent (A) and the colorant (B) may contain either identical or different organic silicon compounds of formula (I) and/or (II).

A process which is particularly preferred in the context of a further version is a process which is exemplified in that the colorant (B) contains at least one organic silicon compound of the formula (I).

R₁R₂N-L-Si(OR₃)ₐ(R₄)_{b} (I),

where
R₁, R₂ both represent a hydrogen atom, and
L represents a linear, divalent C₁-C₆ alkylene group, preferably a propylene group (—CH₂—CH₂—CH₂—) or an ethylene group (—CH₂—CH₂—).

In the context of a further embodiment, a process is particularly preferred in which in the organic silicon compound of the formula (I)

R₁R₂N-L-Si(OR₃)ₐ(R₄)_{b} (I),

R₁, R₂ both represent a hydrogen atom, and

L represents a linear, divalent C₁-C₆ alkylene group, preferably a propylene group (—CH₂—CH₂—CH₂—) or an ethylene group (—CH₂—CH₂—).

A process which is particularly preferred in the context of a further version is a process in which the colorant (B) contains at least one organic silicon compound of the formula (I).

R₁R₂N-L-Si(OR₃)ₐ(R₄)_{b} (I),

where
R₃, R₄ independently of one another represent a methyl group or an ethyl group and
a stands for the number 3 and
b stands for the number 0.

In the context of a further embodiment, a process is particularly preferred in which in the organic silicon compound of the formula (I)

R₁R₂N-L-Si(OR₃)ₐ(R₄)_{b} (I),
R₃, R₄ independently of one another represent a methyl group or an ethyl group and
a stands for the number 3 and
b stands for the number 0.

In a further preferred embodiment, a process as contemplated herein is exemplified in that the colorant (B) contains at least one organic silicon compound of the formula (I) which is selected from the group of
(3-aminopropyl)trimethoxysilane
(3-aminopropyl)triethoxysilane
(2-aminoethyl)trimethoxysilane
(2-aminoethyl)triethoxysilane
(3-dimethylaminopropyl)trimethoxysilane
(3-dimethylaminopropyl)triethoxysilane
(2-dimethylaminoethyl)trimethoxysilane and/or
(2-dimethylaminoethyl)triethoxysilane.

In the context of a further version, a process is particularly preferred in which the organic silicon compound of the formula (I) is selected from the group of
(3-aminopropyl)trimethoxysilane
(3-aminopropyl)triethoxysilane
(2-aminoethyl)trimethoxysilane
(2-aminoethyl)triethoxysilane
(3-dimethylaminopropyl)trimethoxysilane
(3-dimethylaminopropyl)triethoxysilane
(2-dimethylaminoethyl)trimethoxysilane and/or
(2-dimethylaminoethyl)triethoxysilane.

The colorant (B) may also contain at least one organic silicon compound of formula (II).

In a further preferred embodiment, a process as contemplated herein is exemplified in that the colorant (B) contains at least one organic silicon compound corresponding to formula (II).

(R₅O)ₑ(R₆)_{d}Si-(A)ₑ-[NR₇-(A′)]_{f}—[O-(A′″)]_{g}—[NR₈-(A′″)]ₕ—Si(R₆′)_{d′}(OR₅′)_{e′} (II),

where
R5 and R5′ independently represent a methyl group or an ethyl group,
c and c′ both stand for the number 3 and
d and d′ both stand for the number 0

In the context of a further version, a process is particularly preferred in which in the organic silicon compound of the formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
R5 and R5′ independently represent a methyl group or an ethyl group,
c and c′ both stand for the number 3 and
d and d′ both stand for the number 0

In a further preferred embodiment, a process as contemplated herein is exemplified in that the colorant (B) contains at least one organic silicon compound corresponding to formula (II), where
e and f both stand for the number 1,
g and h both stand for the number 0,
A and A′ independently of one another represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In the context of a further version, a process is particularly preferred in which in the organic silicon compound of the formula (II)
e and f both stand for the number 1,
g and h both stand for the number 0,
A and A′ independently of one another represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In a further preferred embodiment, a process as contemplated herein is exemplified in that the colorant (B) contains at least one organic silicon compound corresponding to formula (II), where
e and f both stand for the number 1,
g and h both stand for the number 0,
A and A′ independently of one another represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In a further preferred embodiment, a process as contemplated herein is exemplified in that the colorant (B) contains at least one organic silicon compound corresponding to formula (II), where
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R7 represents a hydrogen atom, a methyl group, a 2-hydroxyethyl group, a 2-alkenyl group, a 2-aminoethyl group or a group of formula (III).

In a further preferred embodiment, a process as contemplated herein is exemplified in that the colorant (B) contains at least one organic silicon compound of the formula (II) which is selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

In the context of a further version, a process is particularly preferred in which the organic silicon compound of formula (II) is selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

The organic silicon compounds of formula (I) and (II) are reactive compounds. In this context, it has proved to be particularly preferred if the colorant (B)—based on the total weight of the colorant (B)—contains one or more organic silicon compounds corresponding to formula (I) and/or (II) in a total quantity of from about 0.1 to about 90.0% by weight, preferably from about 1.0 to about 80.0% by weight, more preferably from about 5.0 to about 50.0% by weight and most preferably from about 10.0 to about 30.0% by weight.

In a further preferred embodiment, a process as contemplated herein is exemplified in that the colorant (B) contains—based on the total weight of the colorant (B)—one or more organic silicon compounds of the formula (I) and/or (II) in a total amount of from about 0.1 to about 90.0% by weight, preferably from about 1.0 to about 80.0% by weight, more preferably from about 5.0 to about 50.0% by weight and very particularly preferably from about 10.0 to about 30.0% by weight.

It has proved to be particularly advantageous to use at least one organic silicon compound of formula (I) in the colorant (B).

In a further preferred embodiment, a process as contemplated herein is exemplified in that the colorant (B) contains at least one organic silicon compound of formula (I).

Furthermore, it has also proved to be particularly suitable to use at least one organic silicon compound of formula (II) in the colorant (B).

In a further preferred embodiment, a process as contemplated herein is exemplified in that the colorant (B) contains at least one organic silicon compound corresponding to formula (II).

In addition to the organic silicon compound(s) of formula (I) and/or (II), the colorant (B) additionally contains at least one colorant compound from the group of direct dyes and/or pigments.

Direct dyes are dyes that are applied directly to the hair and do not require an oxidative process to develop the color. Direct dyes are usually nitrophenylenediamines, nitroaminophenols, azo dyes, anthraquinones, triarylmethane dyes or indophenols.

Direct dyes can be divided into anionic, cationic, and non-ionic direct dyes, which are selected and used by the professional according to the requirements of the carrier base.

In a further preferred embodiment, a process as contemplated herein is exemplified in that the colorant (B) contains at least one direct dye which is preferably selected from the group of anionic, cationic, and non-ionic direct dyes.

Preferred anionic direct dyes are the compounds known under the international names or trade names Bromphenol Blue, Tetrabromophenol Blue, Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1 and Acid Black 52.

Preferred cationic direct dyes are Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Yellow 87, Basic Orange 31 and Basic Red 51.

Non-ionic direct dyes are particularly suitable as non-ionic direct dyes, such as nitro and quinone dyes and neutral azo dyes. The preferred non-ionic direct dyes are those under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

It is also as contemplated herein if the colorant (B) contains as a direct dye at least one dye from the group of thermochromic and/or photochromic dyes.

In a further preferred embodiment, a process as contemplated herein is exemplified in that the colorant (B) contains at least one direct dye which is preferably selected from the group of anionic, cationic, and non-ionic direct dyes.

The best results were obtained when a colorant (B) containing at least one anionic direct dye was used in the process as contemplated herein.

In another particularly preferred embodiment, a process as contemplated herein is exemplified in that the colorant (B) contains at least one anionic direct dye.

The direct dyes may be used in a quantity of from about 0.001 to about 20% by weight, from about 0.05 to about 5% by weight, each based on the total weight of the coloring agent (B). The total amount of direct dyes in the colorant (B) is preferably not more than about 5% by weight.

Pigments within the meaning of the present disclosure are coloring compounds which have a solubility in water at 20° C. of less than about 0.1 g/l. Water solubility can be determined, for example, by the method described below: about 0.1 g of the pigment is weighed in a beaker. A stir-fish is added. Then make up to about 11 with distilled water (20° C.). It is stirred for an hour. If undissolved components of the pigment are still visible in the mixture after this period, the solubility of the pigment is below about 0.1 g/l.

Suitable color pigments may be of organic and/or inorganic origin. Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. In particular, preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

As contemplated herein, colored pearlescent pigments are also particularly preferred color pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, a process as contemplated herein is exemplified in that the colorant (B) contains at least one pigment which is preferably selected from the group of colored metal oxides, metal hydroxides, metal oxide hydrates, silicates, metal sulfides, complex metal cyanides, metal sulfates, bronze pigments and/or from colored pigments based on mica or mica which are coated with at least one metal oxide and/or one metal oxychloride.

In a further preferred embodiment, a process as contemplated herein is exemplified in that the colorant (B) contains at least one pigment selected from pigments based on mica or mica which are combined with one or more metal oxides from the group of titanium dioxide (CI 77891), are coated with black iron oxide (CI 77499), yellow iron oxide (CI 77492), red and/or brown iron oxide (CI 77491, CI 77499), manganese violet (CI 77742), ultramarine (sodium aluminum sulfo-silicates, CI 77007, pigment blue 29), chromium oxide hydrate (CI 77289), chromium oxide (CI 77288) and/or iron blue (ferric ferrocyanides, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491(Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491(Iron oxides)

### Colorona Blackstar Gold, Merck, MICA, CI 77499 (IRON OXIDES)

Other particularly preferred color pigments with the trade name Xirona® are for example

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

Due to their excellent light and temperature resistance, the use of the inorganic color pigments in the agents of the present disclosure is particularly preferred. It is also preferred if the pigments used have a certain particle size. This particle size leads on the one hand to an even distribution of the pigments in the formed polymer film and on the other hand avoids a rough hair or skin feeling after application of the cosmetic product. It is therefore advantageous as contemplated herein if the at least one pigment has an average particle size D₅₀ of from about 1.0 to about 50 μm, preferably of from about 5.0 to about 45 μm, preferably of from about 10 to about 40 μm, of from about 14 to about 30 μm. The mean particle size D₅₀, for example, can be determined using dynamic light scattering (DLS).

The pigment or pigments may be used in an amount of from about 0.001 to about 20% by weight, of from about 0.05 to about 5% by weight, each based on the total weight of the colorant (B).

### Other Ingredients

The pretreatment agent (A) and/or the colorant (B) may also contain one or more surfactants.

The term surfactants refer to surface-active substances. A distinction is made between anionic surfactants of a hydrophobic residue and a negatively charged hydrophilic head group, amphoteric surfactants, which carry both a negative and a compensating positive charge, cationic surfactants, which in addition to a hydrophobic residue have a positively charged hydrophilic group, and nonionic surfactants, which have no charges but strong dipole moments and are strongly hydrated in aqueous solution.

Zwitterionic surfactants are those surface-active compounds which carry at least one quaternary ammonium group and at least one —COO⁽⁻⁾— or —SO₃⁽⁻⁾ group in the molecule. Particularly suitable zwitterionic surfactants are the so-called betaines such as the N-alkyl-N,N-dimethylammonium-glycinate, for example the cocoalkyl-dimethylammoniumglycinate, N-acylaminopropyl-N,N-dimethylammoniumglycinate, for example the cocoacylaminopropyl dimethyl ammonium glycinate, and 2-alkyl-3-carboxymethyl-3-hydroxyethyl imidazolines each having from about 8 to about 18 C atoms in the alkyl or acyl group, and the cocoacylaminoethyl hydroxyethyl carboxymethyl glycinate. A preferred zwitterionic surfactant is the fatty acid amide derivative known under the INCI designation cocamidopropyl betaine.

Ampholytic surfactants are surface-active compounds which, apart from a C₈-C₂₄ alkyl or acyl group, contain at least one free amino group and at least one —COOH— or —SO₃H group in the molecule and can form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about 8 to about 24 C atoms in the alkyl group. Typical examples of amphoteric or zwitterionic surfactants are alkylbetaines, alkylamidobetaines, amino propionates, aminoglycinate, imidazoliniumbetaines and sulfobetaines.

Particularly preferred ampholytic surfactants are the N-cocoalkylaminopropionate, the cocoacylaminoethylaminopropionate and the C₁₂-C₁₈ acylsarcosine.

The pretreatment agent (A) and/or the colorant (B) may additionally contain at least one non-ionic surfactant. Suitable non-ionic surfactants have been shown to be alkyl polyglycosides and alkylene oxide adducts to fatty alcohols and fatty acids with from about 2 to about 30 mol ethylene oxide per mol fatty alcohol or fatty acid. Preparations with good properties are also obtained if they contain as nonionic surfactants fatty acid esters of ethoxylated glycerol which have been reacted with at least 2 mol ethylene oxide. The nonionic surfactants are used in a total quantity of from about 0.1 to about 45% by weight, preferably from about 1 to about 30% by weight and very preferably from about 1 to about 15% by weight—based on the total weight of the respective agent.

In addition, the pretreatment agent (A) and/or the colorant (B) may also contain at least one cationic surfactant. Cationic surfactants are surfactants, i.e. surface-active compounds, each with one or more positive charges. Cationic surfactants contain only positive charges. Usually these surfactants are composed of a hydrophobic part and a hydrophilic head group, the hydrophobic part usually of a hydrocarbon backbone (e.g. of one or two linear or branched alkyl chains) and the positive charge(s) being located in the hydrophilic head group. Examples of cationic surfactants are
quaternary ammonium compounds which, as hydrophobic radicals, may carry one or two alkyl chains with a chain length of from about 8 to about 28 C atoms,
quaternary phosphonium salts substituted with one or more alkyl chains with a chain length of from about 8 to about 28 C atoms or
tertiary sulfonium salts.

Furthermore, the cationic charge can also be part of a heterocyclic ring (e.g. an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total quantity of from about 0.1 to about 45% by weight, preferably from about 1 to about 30% by weight and very preferably from about 1 to about 15% by weight—based on the total weight of the respective agent.

Furthermore, the pretreatment agent (A) and/or the colorant (B) may also contain at least one anionic surfactant. Anionic surfactants are surface-active agents with exclusively anionic charges (neutralized by a corresponding counter cation). Examples of anionic surfactants are fatty acids, alkyl sulfates, alkyl ether sulfates and ether carboxylic acids with from about 12 to about 20 C atoms in the alkyl group and up to 16 glycol ether groups in the molecule.

The anionic surfactants are used in a total quantity of from about 0.1 to about 45% by weight, preferably from about 1 to about 30% by weight and very preferably from about 1 to about 15% by weight—based on the total weight of the respective agent.

The pretreatment agent (A) and/or the colorant (B) may also contain other active substances, auxiliaries and additives, such as solvents, fatty components such as C₅-C₃₀ fatty alcohols, C₈-C₃₀ fatty acid triglycerides, C₈-C₃₀ fatty acid monoglycerides, C₈-C₃₀ fatty acid diglycerides and/or hydrocarbons; nonionic polymers such as vinylpyrrolidinone/vinyl acrylate copolymers, polyvinylpyrrolidinone, vinylpyrrolidinone/vinyl acetate copolymers, polyethylene glycols and polysiloxanes; additional silicones such as volatile or non-volatile, straight-chain, branched or cyclic, crosslinked or non-crosslinked polyalkylsiloxanes (such as dimethicones or cyclomethicones), polyarylsiloxanes and/or polyalkylarylsiloxanes, in particular polysiloxanes with organofunctional groups, such as substituted or unsubstituted amines (amodimethicones), carboxyl, alkoxy and/or hydroxyl groups (dimethicone copolymers), linear polysiloxaneA)-polyoxyalkyleneB)-block copolymers, grafted silicone polymers; cationic polymers such as quaternized cellulose ethers, polysiloxanes having quaternary groups, dimethyldiallylammonium chloride polymers, acrylamide-dimethyldiallylammonium chloride copolymers, dimethylamino-ethylmethacrylate-vinylpyrrolidinone copolymers quaternized with diethyl sulfate, vinylpyrrolidinone-imidazolinium-methochloride copolymers and quaternized polyvinyl alcohol; zwitterionic and amphoteric polymers; anionic polymers such as polyacrylic acids or crosslinked polyacrylic acids; structural agents such as glucose, maleic acid and lactic acid; hair conditioning compounds such as phospholipids, for example lecithin and cephalins; perfume oils, dimethylisosorbide and cyclodextrins; fiber structure-improving active substances, in particular mono-, di- and oligosaccharides such as glucose, galactose, fructose, fructose and lactose; dyes for coloring the composition; anti-dandruff active substances such as Piroctone Olamine, Zinc Omadine and Climbazol; amino acids and oligopeptides; protein hydrolysates on an animal and/or vegetable basis, as well as in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetrating substances such as glycerol, propylene glycol monoethyl ether, carbonates, hydrogen carbonates, guanidines, ureas as well as primary, secondary and tertiary phosphates; opacifiers such as latex, styrene/PVP and styrene/acrylamide copolymers; pearlescent agents such as ethylene glycol mono- and distearate and PEG-3 distearate; and blowing agents such as propane-butane mixtures, N₂O, dimethyl ether, CO₂ and air.

The selection of these additional substances will be made by the specialist according to the desired properties of the agents. Regarding other optional components and the quantities of these components used, explicit reference is made to the relevant manuals known to the specialist. The additional active ingredients and auxiliary substances are preferably used in the preparations as contemplated herein in amounts of from about 0.0001 to about 25% by weight in each case, from about 0.0005 to about 15% by weight, based on the total weight of the respective preparation.

### Procedural Steps

The technical application properties of the resulting dyeing can be further improved by selecting the optimum process conditions.

In another preferred form of execution, a process comprising the following steps in the order indicated is particularly preferred

(1) Apply pretreatment agent (A) to the hair,
(2) Allow the pretreatment agent (A) to act on the hair,
(3) if necessary, rinse out the pre-treatment agent (A),
(4) Apply the dye (B) to the hair,
(5) Let the dye (B) act on the hair and
(6) Rinse out the hair.

In a first step (1), the pre-treatment agent (A) is applied to the hair.

After application, the pre-treatment agent (A) can act on the hair. In this context, application times from about 10 seconds to about 10 minutes, preferably from about 20 seconds to about 5 minutes and especially preferably from about 30 seconds to about 2 minutes on the hair have proven to be particularly beneficial.

In a further preferred embodiment, a process as contemplated herein is exemplified by

(2) Allow the pretreatment agent (A) to act on the hair for a period of from about 10 seconds to about 10 minutes, preferably from about 10 seconds to about 5 minutes and most preferably from about 30 seconds to about 2 minutes.

In a preferred embodiment of the method as contemplated herein, the pretreatment agent (A) can now be rinsed from the hair before the colorant (B) is applied to the hair in the subsequent step.

In a further embodiment, a process comprising the following steps in the order given is particularly preferred

(1) Apply pretreatment agent (A) to the hair,
(2) Allow the pretreatment agent (A) to act on the hair,
(3) Rinse out the pretreatment agent (A),
(4) Apply the dye (B) to the hair,
(5) Let the dye (B) act on the hair and
(6) Rinse out the hair.

Dyeing's with also good wash fastness properties were obtained when the dye (B) was applied to the hair which was still treated with the pretreatment agent (A).

In a further embodiment, a process comprising the following steps in the order given is particularly preferred

(1) Apply pretreatment agent (A) to the hair,
(2) Allow the pretreatment agent (A) to act on the hair,
(3) no rinsing of the pretreatment agent (A),
(4) Apply the dye (B) to the hair,
(5) Let the dye (B) act on the hair and
(6) Rinse out the hair.

In step (4) the dye (B) is now applied to the hair. After application, let the dye (B) act on the hair.

The process as contemplated herein allows the production of dyeing's with particularly good intensity and wash fastness even with a short exposure time of the dye (B). Application times from about 10 seconds to about 10 minutes, preferably from about 20 seconds to about 5 minutes and most preferably from about 30 seconds to about 3 minutes on the hair have proven to be particularly beneficial.

In a further preferred embodiment, a process as contemplated herein is exemplified by

(5) Allow the dye (B) to act on the hair for a period of from about 10 seconds to about 10 minutes, preferably from about 20 seconds to about 5 minutes and most preferably from about 30 seconds to about 3 minutes.

In another preferred form of execution, a process comprising the following steps in the order indicated is particularly preferred

(1) Apply pretreatment agent (A) to the hair,
(2) Allow the pretreatment agent (A) to act on the hair for a period of from about 20 seconds to about 5 minutes,
(3) if necessary, rinse out the pre-treatment agent (A),
(4) Apply the dye (B) to the hair,
(5) Allow the dye (B) to act on the hair and for a period of from about 20 seconds to about 5 minutes and
(6) Rinse out the hair.

The pretreatment agent (A) and the colorant (B) both contain reactive organic silicon compounds of formula (I) and/or (II), which form a film on the hair. In order to obtain a film that is as homogeneous and resistant as possible, it has been found to be particularly preferable if there is a period of maximum about 48 hours, preferably maximum about 24 hours, more preferably maximum about 12 hours and most preferably maximum about 6 hours between the application of the pre-treatment agent (A) and the application of the coloring agent (B).

In a further preferred embodiment, a process as contemplated herein is exemplified in that the pretreatment agent (A) and the colorant (B) are applied to the hair within a period of at most about 48 hours, preferably at most about 24 hours, more preferably at most about 12 hours and most preferably at most about 6 hours.

### Multi-Component Packaging Unit (Kit-of-Parts)

In carrying out the process as contemplated herein, the user applies the agents described above, the pretreatment agent (A) and the colorant (B). Both agents are, as already mentioned, the ready-to-use agents.

It is particularly convenient for the user if all components or preparations required to produce the pretreatment agent (A) and the colorant (B) are provided in the form of a multi-component packaging unit.

The organic silicon compounds of formula (I) and (II) are reactive compounds. In order to avoid undesired, premature reactions and incompatibilities, it may be particularly preferable within the framework of a design form if the user himself prepares the ready-to-use pretreatment agent (A) and/or the ready-to-use colorant (B) by mixing different preparations only shortly before use.

For this purpose, a multi-component packaging unit as contemplated herein may also contain one or more additional agents in addition to the separately assembled agents (A) and (B).

Separate storage of the organic silicon compounds of formulae (I) or (II) and the coloring compounds has proven to be advantageous in this context.

A second object of the present disclosure is therefore a kit-of-parts for coloring human hair, comprising separately packaged
a first container with an agent (A) which contains at least one organic silicon compound of the formula (I) and/or (II) and contains no direct dyes and no pigments, and
a second container with an agent (B) containing at least one organic silicon compound of formula (I) and/or (II), and
where appropriate, a third container with a medium (B′),
   wherein at least one of the agents (B) and/or (B′) contains at least one colorant compound from the group of direct dyes and pigments, wherein in the organic silicon compound of formula (I)

R₁R₂N-L-Si(OR₃)ₐ(R4)_{b} (I),
R₁, R₂ independently of one another represent a hydrogen atom or a C₁-C₆ alkyl group,
L is a linear or branched divalent C₁-C₂₀ alkylene group,
R₃, R₄ independently of one another represent a C₁-C alkyl group,
a, is an integer from 1 to 3, and
b stands for the integer 3−a, and
   wherein in the organic silicon compound of formula (II)

(R₅O)ₑ(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
R5, R5 ‘, R5″, R6, R6’ and R6″ independently of one another represent a C₁-C₆ alkyl group,
A, A′, A″, A′″ and A″″ independently of one another represent a linear or branched divalent C₁-C₂₀ alkylene group
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
provided that at least one of e, f, g, and h is different from 0.

In the context of an execution form, agent (A) is equal to the pre-treatment agent (A) ready for use. However, it is preferable if the pre-treatment agent (A) is prepared from agent (A) shortly before application. This can be done, for example, by mixing agent (A), which contains the organic silicon compound of formula (I) and/or (II) and is low in water or in anhydrous form, with another agent (A′) containing water.

Similarly, in the context of a design form, the agent (B) can be the ready-to-use colorant (B). However, it is also preferred if the agent (B) is used to produce the ready-to-use colorant (B) shortly before application. This can be done, for example, by mixing agent (B), which contains the organic silicon compound of formula (I) and/or (II) and is low in water or in anhydrous form, with another agent (B′) containing water.

A preferred embodiment is therefore a multi-component packaging unit (kit-of-parts) for dyeing human hair, which is made up separately from one another
a first container with an essentially anhydrous agent (A) which contains at least one organic silicon compound of the formula (I) and/or (II) and furthermore contains no substantive dyes and no pigments, and
a second container with an aqueous agent (A′), which contains no direct dyes and no pigments and
a third container containing a substantially anhydrous agent (B) containing at least one organic silicon compound of formula (I) and/or (II), and
a fourth container containing an aqueous agent (B′),
   wherein at least one of the agents (B) and/or (B′) contains at least one colorant compound from the group of direct dyes and pigments, and
   wherein the organic silicon compound of formula (I) and/or (II) represent the compounds described above.

By mixing agents (A) and (A′) the ready-to-use pre-treatment agent (A) is produced. By mixing agents (B) and (B′) the ready-to-use colorant (B) is produced.

A multi-component packaging unit is particularly preferred within the framework of a further design form, in which the agent (A) is essentially anhydrous and preferably—based on the total weight of the agent (A)—contains less than about 5.0% by weight, more preferably less than about 2.5% by weight, still more preferably less than about 1.0% by weight and very particularly preferably less than about 0.1% by weight of water.

A multi-component packaging unit is particularly preferred within the framework of a further design form, in which the agent (B) is substantially anhydrous and preferably—based on the total weight of the agent (B)—contains less than about 5.0% by weight, more preferably less than about 2.5% by weight, still more preferably less than about 1.0% by weight and very particularly preferably less than about 0.1% by weight of water.

Agents (A) and/or (B) can be in the form of a powder, for example. It is also possible to make the agents (A) and/or (B) available as paste or oil. In this case, the organic silicon compounds of formula (I) may be incorporated into an inert cosmetic carrier. Fatty components have proven to be suitable as inert cosmetic carriers.

As contemplated herein, fat constituents are organic compounds with a solubility in water at room temperature (22° C.) and atmospheric pressure (760 mmHg) of less than about 1% by weight, preferably less than about 0.1% by weight. The definition of fat constituents explicitly covers only uncharged (i.e. non-ionic) compounds. Fatty components have at least one saturated or unsaturated alkyl group with at least 8 C atoms. The molecular weight of the fat constituents is a maximum of about 5000 g/mol, preferably a maximum of about 2500 g/mol and particularly preferably a maximum of about 1000 g/mol. The fat components are neither polyoxyalkylated nor polyglycerylated compounds.

In this context, preferred fat constituents are defined as the constituents from the group of C₅-C₃₀ fatty alcohols, C₅-C₃₀ fatty acid triglycerides, C₅-C₃₀ fatty acid monoglycerides, C₅-C₃₀ fatty acid diglycerides and/or hydrocarbons. For the purposes of the present disclosure, only non-ionic substances are explicitly regarded as fat components. Charged compounds such as fatty acids and their salts are not considered to be fat components.

C₅-C₃₀ fatty alcohols can be saturated, mono- or polyunsaturated, linear, or branched fatty alcohols with from about 8 to about 30 C atoms.

Examples of preferred C₈-C₃₀ linear saturated fatty alcohols are dodecan-1-ol (dodecyl alcohol, lauryl alcohol), tetradecan-1-ol (tetradecyl alcohol, myristyl alcohol), hexadecan-1-ol (hexadecyl alcohol), cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear unsaturated fatty alcohols are (9Z)-octadec-9-en-1-ol(oleyl alcohol), (9E)-octadec-9-en-1-ol(elaidyl alcohol) (9Z,12Z)-octadeca-9,12-dien-1-ol (linoleyl alcohol), (9Z,12Z,15Z)-octadeca-9,12, 15-trien-1-ol (linolenoyl alcohol), gadoleyl alcohol ((9Z)-Eicos-9-en-1-ol), arachidone alcohol ((5Z,8Z,11Z,14Z)-Eicosa-5,8,11,14-tetraen-1-ol), erucyl alcohol ((13Z)-Docos-13-en-1-ol) and/or brassidyl alcohol ((13E)-Docosen-1-ol).

The preferred representatives for branched fatty alcohols are 2-octyl-dodecanol, 2-hexyl-dodecanol and/or 2-butyl-dodecanol.

For the purposes of this present disclosure, a C₈-C₃₀ fatty acid triglyceride is understood to be the triester of trivalent alcohol glycerol with three equivalents of fatty acid. Both structurally identical and different fatty acids within a triglyceride molecule can be involved in the formation of esters.

As contemplated herein, fatty acids are saturated or unsaturated, unbranched, or branched, unsubstituted, or substituted C₈-C₃₀ carboxylic acids. Unsaturated fatty acids can be mono- or polyunsaturated. For an unsaturated fatty acid, its C═C double bond(s) may have the Cis or Trans configuration.

Fatty acid triglycerides are exemplified by their particular suitability, in which at least one of the ester groups is formed from glycerol with a fatty acid selected from dodecanoic acid (lauric acid), Tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], Palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-cosa-5,8,11,14-tetraenoic acid] and/or nervonic acid [(15Z)-tetracos-15-enoic acid].

The fatty acid triglycerides can also be of natural origin. The fatty acid triglycerides or mixtures thereof occurring in soybean oil, peanut oil, olive oil, sunflower oil, macadamia nut oil, moringa oil, apricot kernel oil, marula oil and/or optionally hardened castor oil are particularly suitable for use in the product as contemplated herein.

A C₈-C₃₀ fatty acid monoglyceride is the monoester of the trivalent alcohol glycerol with one equivalent of fatty acid. Either the middle hydroxy group of glycerol or the terminal hydroxy group of glycerol may be esterified with the fatty acid.

The C₅-C₃₀ fatty acid monoglyceride is exemplified by its special suitability, in which a hydroxy group of the glycerol is esterified with a fatty acid, whereby the fatty acids are selected from dodecanoic acid (lauric acid), Tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], Palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-cosa-5,8,11,14-tetraenoic acid] or nervonic acid [(15Z)-tetracos-15-enoic acid].

A C₅-C₃₀ fatty acid diglyceride is the diester of the trivalent alcohol glycerol with two equivalents of fatty acid. Either the middle and one terminal hydroxy group of glycerol may be esterified with two equivalents of fatty acid, or both terminal hydroxy groups of glycerol are esterified with one fatty acid each. The glycerol can be esterified with two structurally identical fatty acids or with two different fatty acids.

Fatty acid diglycerides are exemplified by their special suitability, in which at least one of the ester groups is formed from glycerol with a fatty acid selected from dodecanoic acid (lauric acid), Tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], Palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid, linolenic acid [(9Z, 12Z,15Z)-octadeca-9,12,15-trienoic acid, elaeostearic acid [(9Z, 11E,13E)-octadeca-9, 11,3-trienoic acid], arachidonic acid [(5Z,8Z,11Z,14Z)-cosa-5,8,11,14-tetraenoic acid] and/or nervonic acid [(15Z)-tetracos-15-enoic acid].

As contemplated herein, the term C₅-C₃₀ fatty acid esters refers to esters of C₅-C₃₀ fatty acids with aliphatic C₁-C₁₀ alcohols. The C₁-C₁₀ alcohols can be linear. From a chain length of 3 C atoms, the alcohols can also be branched.

Particularly suitable C₈-C₃₀ fatty acid esters can be selected from the group of isopropyl myristate (myristic acid isopropyl ester) and isopropyl stearate (stearic acid isopropyl ester).

Hydrocarbons are compounds consisting exclusively of the atoms carbon and hydrogen with from about 8 to about 80 C atoms. In this context, aliphatic hydrocarbons such as mineral oils, liquid paraffin oils (e.g. Paraffinium Liquidum or Paraffinum Perliquidum), isoparaffin oils, semi-solid paraffin oils, paraffin waxes, hard paraffin (Paraffinum Solidum), Vaseline and polydecenes are particularly preferred.

Liquid paraffin oils (Paraffinum Liquidum and Paraffinium Perliquidum) have proven to be particularly suitable in this context. Paraffinum Liquidum, also known as white oil, is the preferred hydrocarbon. Paraffinum Liquidum is a mixture of purified, saturated, aliphatic hydrocarbons, including mainly hydrocarbon chains with a C-chain distribution of from about 25 to about 35 C-atoms.

A multi-component packaging unit is particularly preferred in the context of another version, exemplified in that the cosmetic preparation (A) contains one or more fat constituents from the group of C₈-C₃₀ fatty alcohols, C₈-C₃₀ fatty acid triglycerides, C₈-C₃₀ fatty acid monoglycerides, C₈-C₃₀ fatty acid diglycerides, C₈-C₃₀ fatty acid esters and/or hydrocarbons.

It has been found that C₈-C₃₀ fatty alcohols and hydrocarbons are particularly suitable as inert cosmetic carriers.

A multi-component packaging unit is particularly preferred within the framework of a further version, in which the agent (A) contains one or more fat constituents from the group of C₅-C₃₀ fatty alcohols and/or hydrocarbons.

A multi-component packaging unit is particularly preferred within the framework of a further version, in which the agent (B) contains one or more fat constituents from the group of C₅-C₃₀ fatty alcohols and/or hydrocarbons.

A multi-component packaging unit is particularly preferred within the framework of a further design form, in which the agent (A′)—based on the total weight of the agent (A′)—contains from about 50 to about 100% by weight, preferably from about 50 to about 95% by weight, more preferably from about 60 to about 95% by weight and very particularly preferably from about 70 to about 95% by weight of water.

A multi-component packaging unit is particularly preferred within the framework of a further design form, in which the agent (B′)—based on the total weight of the agent (B′)—contains from about 50 to about 100% by weight, preferably from about 50 to about 95% by weight, more preferably from about 60 to about 95% by weight and very particularly preferably from about 70 to about 95% by weight of water.

In addition, the aqueous agents (A′) and/or (B′) may also contain at least one acid. Suitable acids can be selected from the organic and inorganic acids. Suitable organic acids are lactic acid, citric acid, tartaric acid, malic acid, 1-hydroxyethane-1,1-diphosphonic acid, 2,6-dipicolinic acid, benzoic acid, maleic acid, succinic acid, oxalic acid, ascorbic acid, phytic acid and/or gluconic acid. Suitable inorganic acids are phosphoric acid, sulfuric acid, and hydrochloric acid.

A multi-component packaging unit in which the agent (A′) contains at least one acid from the group comprising lactic acid, citric acid, tartaric acid, malic acid, 1-hydroxyethane-1,1-diphosphonic acid, 2,6-dipicolinic acid, benzoic acid, phosphoric acid, sulfuric acid, hydrochloric acid, maleic acid, succinic acid, oxalic acid, ascorbic acid, phytic acid and/or gluconic acid.

A multi-component packaging unit in which the agent (B′) contains at least one acid from the group lactic acid, citric acid, tartaric acid, malic acid, 1-hydroxyethane-1.1-diphosphonic acid, 2,6-dipicolinic acid, benzoic acid, phosphoric acid, sulfuric acid, hydrochloric acid, maleic acid, succinic acid, oxalic acid, ascorbic acid, phytic acid and/or gluconic acid is particularly preferred within the framework of a further design form.

With respect to the other preferred embodiments of the multi-component packaging unit as contemplated herein and the organosilicon compounds of formulae (I) and/or (II), the procedure said for the procedure as contemplated herein applies mutatis mutantis.

### Examples

### 1. Formulations

The following formulations were produced.

| Medium (A) | (A) |
|---|---|
| (3-Aminopropyl)triethoxysilan | 2.0 g |

| Medium (A′) | (A′) | |
|---|---|---|
| Cocoamidopropyl betaine | 1.5 | g |
| Citric acid | ad pH | 3.5 |
| Lactic acid | — | |
| Sulfuric acid | — | |
| Water | ad 98 | g |

By mixing 2 g of agent (A) and 98 g of agent (A′) the pre-treatment agent (A) was prepared ready for use. During this procedure, agents (A) and (A′) were shaken together for 3 minutes. Then the pretreatment agent (A) was left to stand for about 5 minutes and then applied to hair strands.

| Medium (B) | (B) |
|---|---|
| (3-Aminopropyl)triethoxysilan | 20.0 g |

| Medium (B′) | (B′) | |
|---|---|---|
| Cocoamidopropyl betaine | 1.5 | g |
| Ext. D&C Violet 2 | 1.0 | g |
| (Acid Violet 43, CI 60730, CAS-No. 4430-18-6) | | |
| Citric acid | ad pH | 3.5 |
| Water | ad 80 | g |

By mixing 20 g of agent (B) and 80 g of agent (B′) the ready-to-use dye (B) was prepared. During this procedure, agents (B) and (B′) were each shaken together for 3 minutes. Then the dye (B) was left to stand for about 5 minutes and then applied to hair strands.

### 2. Application

The pretreatment agent (A) and the colorant (B) were each applied with a brush to hair strands (Kerling 6-0) and left to act for the specified period.

In example E1, the dye (B) was applied directly after washing out the pretreatment agent (A).

Afterwards the hair strands were first washed out with water and then washed and dried with a shampoo. Afterwards the strands were visually evaluated.

The strands were then washed several times with a shampoo and again visually evaluated.

| | | E1 |
|---|---|---|
| | V1 | according to present |
| | Comparison | disclosure |
| Pretreatment | — | (A) + (A′) |
| agent (A) | | |
| Period of | — | 5 min |
| application of | | |
| Pretreatment | | |
| agent (A) | | |
| Rinsing of | — | yes |
| pretreatment | | |
| agent (A) | | |
| Coloring | (B) + (B′) | (B) + (B′) |
| agent (B) | | |
| Duration of | 5 min | 5 min |
| application of | | |
| colorant (B) | | |
| Rinse out of | yes | yes |
| Coloring agent (B) | | |
| Result | violet | violet |
| | coloration | coloration |
| | ++ | +++ |
| | particularly | particularly |
| | good washability | good washability |

Intensity:
+ bad
++ medium
+++ high

In the comparative example V1 no pre-treatment was carried out. In example E1 the strands were treated with the pretreatment agent (A).

Compared to V1, a further improvement in color intensity was achieved in E1.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Process for dyeing human hair, wherein
a pretreatment agent (A) which comprises at least one organic silicon compound of the formula (I) and/or (II) and comprises no direct dyes and no pigments, and
a colorant (B) which comprises at least one organic silicon compound of the formula (I) and/or (II) and further comprises at least one colorant compound from the group of direct dyes and/or pigments,
is applied to the hair,
wherein in the organic silicon compound of formula (I)
R₁R₂N-L-Si(OR₃)ₐ(R₄)_{b} (I),
R₁, R₂ independently of one another represent a hydrogen atom or a C₁-C₆ alkyl group,
L is a linear or branched divalent C₁-C₂₀ alkylene group,
R₃, R₄ independently of one another represent a C₁-C alkyl group,
a, is an integer from 1 to 3, and
b stands for the integer 3−a, and
wherein in the organic silicon compound of formula (II)
(R₅O)_{c}(R₆)_{d}Si-(A)ₑ-[NR₇-(A′)]_{f}—[O-(A″)[_{g}—[NR₈-(A′″)]ₕ—Si(R₆′)_{d′}(OR₅′)_{c′} (II),
R5, R5′, R5″, R6, R6′ and R6″ independently of one another represent a C₁-C₆ alkyl group,
A, A′, A″, and A′″ independently of one another represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
A″″ represents a linear or branched divalent C₁-C₂₀ alkylene group,
provided that at least one of e, f, g, and h is different from 0.

2. Process according to claim 1, wherein, in the organic silicon compound of the formula (I)
R₁R₂N-L-Si(OR₃)ₐ(R₄)_{b} (I),
R₁, R₂ both represent a hydrogen atom, and
L represents a linear, divalent C₁-C₆ alkylene group.

3. Process according to claim 1, wherein, in the organic silicon compound of the formula (I)
R₃, R₄ independently of one another represent a methyl group or an ethyl group and
a stands for the number 3 and
b stands for the number 0.

4. A process according to claim 1, wherein the organic silicon compound of formula (I) is selected from the group of
(3-aminopropyl)trimethoxysilane,
(3-aminopropyl)triethoxysilane,
(2-aminoethyl)trimethoxysilane,
(2-aminoethyl)triethoxysilane,
(3-dimethylaminopropyl)trimethoxysilane,
(3-dimethylaminopropyl)triethoxysilane,
(2-dimethylaminoethyl)trimethoxysilane, and/or
(2-dimethylaminoethyl)triethoxysilane.

5. Process according to claim 1, wherein in the organic silicon compound of the formula (II)
(R₅O)_{c}(R₆)_{d}Si-(A)ₑ-[NR₇-(A′)]_{f}—[O-(A″)[_{g}—[NR₈-(A′″)]ₕ—Si(R₆′)_{d′}(OR₅′)_{c′} (II),
R5 and R5′ independently represent a methyl group or an ethyl group,
c and c′ both stand for the number 3 and
d and d′ both stand for the number 0.

6. Process according to claim 1, wherein in the organic silicon compound of the formula (II)
e and f both stand for the number 1,
g and h both stand for the number 0,
A and A′ independently of one another represent a linear, divalent C₁-C₆ alkylene group
and
R7 represents a hydrogen atom, a methyl group, a 2-hydroxyethyl group, a 2-alkenyl group, a 2-aminoethyl group or a group of formula (III).

7. A process according to claim 1, wherein the organic silicon compound of formula (II) is selected from the group of
3-(trimethoxysilyl)-N-[3-(trimethoxysilyl)propyl]-1-propanamine,
3-(Triethoxysilyl)-N-[3-(triethoxysilyl)propyl]-1-propanamine,
N-methyl-3-(trimethoxysilyl)-N-[3-(trimethoxysilyl)propyl]-1-propanamine,
N-methyl-3-(triethoxysilyl)-N-[3-(triethoxysilyl)propyl]-1-propanamine,
2-[Bis[3-(trimethoxysilyl)propyl]amino]-ethanol,
2-[bis[3-(triethoxysilyl)propyl]amino]ethanol,
3-(trimethoxysilyl)-N,N-bis[3-(trimethoxysilyl)propyl]-1-propanamine,
3-(Triethoxysilyl)-N,N-bis[3-(triethoxysilyl)propyl]-1-propanamine,
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

8. Process according to claim 1, wherein the pretreatment agent (A) comprises—based on the total weight of the pretreatment agent (A)—one or more organic silicon compounds of the formula (I) and/or (II) in a total amount of from about 0.1 to about 20.0% by weight.

9. Process according to claim 1, wherein the pretreatment agent (A) comprises at least one organic silicon compound of the formula (I).

10. Process according to claim 1, wherein the colorant (B) comprises—based on the total weight of the colorant (B)—one or more organic silicon compounds corresponding to formula (I) and/or (II) in a total quantity of from about 0.1 to about 90.0% by weight.

11. Process according to claim 1, wherein the colorant (B) comprises at least one organic silicon compound corresponding to formula (I).

12. Process according to claim 1, wherein the colorant (B) comprises at least one organic silicon compound corresponding to formula (II).

13. Process according to claim 1, wherein the colorant (B) comprises at least one direct dye.

14. Process according to claim 1, wherein the colorant (B) comprises at least one pigment.

15. A process according to claim 1, wherein the colorant (B) comprises at least one pigment which is selected from pigments based on mica or mica, which are mixed with one or more metal oxides from the group comprising titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfo-silicate, CI 77007, Pigment Blue 29), chromium oxide hydrate (CI 77289), chromium oxide (CI 77288) and/or iron blue (Ferric Ferrocyanide, CI 77510) are coated.

16. A method according to claim 1, comprising the following steps in the order indicated
(1) applying pretreatment agent (A) to the hair,
(2) allowing the pretreatment agent (A) to act on the hair,
(3) optionally, rinsing out the pre-treatment agent (A),
(4) applying the dye (B) to the hair,
(5) allowing the dye (B) act on the hair and
(6) rinsing out the hair.

17. Process according to claim 16, wherein
(2) the pretreatment agent (A) is allowed to act on the hair for a period of from about 10 seconds to about 10 minutes.

18. Process according to claim 16, wherein
(5) the dye (B) is allowed to act on the hair for a period of from about 10 seconds to about 10 minutes.

19. Process according to claim 1, wherein the pretreatment agent (A) and the colorant (B) are applied to the hair within a period of at most about 48 hours.

20. Multi-component packaging unit for dyeing human hair, comprehensively assembled separately
a first container with an agent (A) which comprises at least one organic silicon compound of the formula (I) and/or (II) and comprises no direct dyes and no pigments, and
a second container with an agent (B) comprising at least one organic silicon compound of formula (I) and/or (II), and
optionally, a third container with a medium (B′),
wherein at least one of the agents (B) and/or (B′) comprises at least one colorant compound from the group of direct dyes and pigments,
wherein in the organic silicon compound of formula (I)
R₁R₂N-L-Si(OR₃)ₐ(R₄)_{b} (I),
R₁, R₂ independently of one another represent a hydrogen atom or a C₁-C₆ alkyl group,
L is a linear or branched divalent C₁-C₂₀ alkylene group,
R₃, R₄ independently of one another represent a C₁-C alkyl group,
a, is an integer from 1 to 3, and
b stands for the integer 3−a, and
wherein in the organic silicon compound of formula (II)
(R₅O)_{c}(R₆)_{d}Si-(A)ₑ-[NR₇-(A′)]_{f}—[O-(A″)[_{g}—[NR₈-(A′″)]ₕ—Si(R₆′)_{d′}(OR₅′)_{c′} (II),
R5, R5′, R5″, R6, R6′ and R6″ independently of one another represent a C₁-C₆ alkyl group,
A, A′, A″, and A′″ independently of one another represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d}″(OR₅″)_{c}″ (III),
c, stands for an integer from 1 to 3,
d stands for the integer 3−c,
c′ stands for an integer from 1 to 3,
d′ stands for the integer 3−c′,
c″ stands for an integer from 1 to 3,
d″ stands for the integer 3−c″,
e stands for 0 or 1,
f stands for 0 or 1,
g stands for 0 or 1,
h stands for 0 or 1,
A″″ represents a linear or branched divalent C₁-C₂₀ alkylene group,
provided that at least one of e, f, g, and h is different from 0.
